(19) United States
(12) Patent Application Publication
(10) Pub. No.: US 2019/0117238 A1
(43) Pub. Date: Apr. 25, 2019

(54) Percutaneous Metatarsal Fixation Guide and Methods of Use

(71) Applicant: Andrew Levitt, Gilbert, AZ (US)

(72) Inventor: Andrew Levitt, Gilbert, AZ (US)

(21) Appl. No.: 16/167,320

(22) Filed: Oct. 22, 2018

Related U.S. Application Data
(60) Provisional application No. 62/575,297, filed on Oct. 20, 2017.

(51) Int. Cl. A61B 17/17; A61B 17/72

(52) U.S. Cl. CPC A61B 17/1775; A61B 17/1725; A61B 2017/681; A61B 17/7233; A61B 17/7291

(57) ABSTRACT

A percutaneous metatarsal fixation guide, including methods of use, is provided. The percutaneous metatarsal fixation guide includes a means for aligning a drill guide bearing a drill sleeve with the central longitudinal axis of a fractured metatarsal, metacarpal, or similar bone. Precise radiographic alignment of the drill sleeve with the long axis of the fractured bone facilitates accurate placement of fixation hardware across the fracture site, allowing for healing in alignment. The fixation bearing two corresponding index wheels is rigidly mounted to a tarsal bone via a temporary threaded K-wire, wherein an index marking on a second index wheel coupled to a drill sleeve may be positioned to correspond with an index marking on first index wheel coupled to an alignment guide, such that the drill sleeve is aligned with the fractured metatarsal. This allows precise placement of fixation hardware, compared to using a conventional “free-hand” technique.

### CROSS REFERENCE TO RELATED APPLICATION

This application claims priority from United States Provisional Patent Application No. 62/575,297 filed Oct. 20, 2017 and entitled “Percutaneous Metatarsal Fixation Guide and Methods of Use,” which is incorporated entirely herein by reference.

### BACKGROUND OF THE INVENTION

### Technical Field

This invention relates to percutaneous fixation guides and systems. Specifically, embodiments of the invention relate to a percutaneous metatarsal fixation guide and methods of use.

### State of the Art

Metatarsal fractures are a common occurrence accounting for 55% of all foot fractures. The mechanism of injury is usually high energy such as a motor vehicle accident (MVA) or simply blunt trauma from an object striking the foot. Non-displaced fractures are treated non-operatively in anything from a stiff soled shoe to a cam-boot or short leg cast depending on the patient. Many authors and research papers recommend reducing metatarsal fractures with displacement of more than 3-4 mm or angulations of more than 10 degrees. Closed reduction of metatarsal fractures via distraction of the corresponding digit is attempted but often doesn't relocate the displaced fragment due to the weak soft tissue attachments & maintenance of the reduction is uncertain without internal fixation (Lindholm). Closed Reduction Percutaneous Pinning (CRPP) is generally attempted in the OR but may not achieve anatomical reduction due to their small size and location within the forefoot preventing a direct firm grasp. This often forces the surgeons hand towards Open Reduction Internal Fixation (ORIF) which may not be desirable in patients with multiple comorbidities, vascular issues, or poor soft tissue envelope. The soft tissue envelope covering the distal extremities is a relatively thin layer of skin, fascia, tendon, & bone which is left unprotected by the extremely vascularized muscles located more proximally up on the leg. This leads to an inherently greater chance of osteomyelitis, infection, & soft tissue complications, Fracture blisters, abrasions, or forefoot edema may delay surgical treatment for 2-3+ weeks to reduce risk of surgical site dehiscence, exposed hardware, & infection associated with ORIF.

Even with meticulous dissection, ORIF causes further disruption to the already traumatized skin, soft tissue, blood supply, & periosteum surrounding the fracture site of the distal extremity. An incision through these compromised structures increases the risk of avascular necrosis, nonunion, scar tissue, tendon & soft tissue contractures. Percutaneous fixation inherently carries less risk since there is no incision.

Placement of percutaneous K-wires “free-handed” is widely practiced but achieving proper alignment is not consistent. Even if the fracture is closed reduced, it typically requires multiple attempts to place the K-wire percutaneously through the skin, across the small fracture site & up the shaft into stable bone. Each additional attempt further weakens the bone & leads to loss of stable fixation, commonly called the “swiss cheese” effect. More often than not the fracture is not adequately closed reduced necessitating open reduction & therefore an incision. However, percutaneous fixation has less benefit if an open approach is required to achieve anatomical reduction.

If anatomic reduction could be achieved percutaneously then definitive percutaneous fixation could be performed immediately. Percutaneous reduction & fixation would also significantly reduce overall recovery time compared to ORIF which may be delayed 2-3+ weeks while soft tissue complications resolve prior to making an open incision. Accordingly, what is needed is a reproducible technique for percutaneous anatomical reduction & an associated device for precise placement of percutaneous fixation in all three dimensions.

### SUMMARY OF THE INVENTION

Embodiments of the present invention include a percutaneous metatarsal fixation guide which is reversibly mounted to a “base pillar.” In some embodiments, the base pillar may be a threaded heavy-gauge Kirshner wire temporarily screwed into the tarsal bone articulating with the base of the fractured metatarsal. The percutaneous metatarsal fixation guide can also be mounted to an external frame, such as an external fixator. The fixation guide mounted to the base pillar, or other fixed point such as an external frame or external fixator, is used by a foot surgeon to precisely align a drill guide sleeve in three orthogonal planes with the long axis of the fractured metatarsal under intraoperative fluoroscopy. Precise placement of fixation hardware through the aligned drill guide sleeve, versus “free-hand” hardware placement, decreases the risk of patient complications from mal-alignment, length shortening, non-union, and the like.

Disclosed is a percutaneous metatarsal fixation guide comprising an arm member having a pillar end and a guide end; a base pillar receiver coupled to the arm member proximate to the pillar end; and a drill guide coupled to the arm member proximate to the guide end.

In some embodiments, the percutaneous metatarsal fixation guide further comprises a coronal swivel interposed between the arm member and the base pillar receiver, wherein the coronal swivel permits rotation of the base pillar receiver with respect to the pillar end. In some embodiments, the coronal swivel is a locking coronal swivel. In some embodiments, the percutaneous metatarsal fixation guide comprises a first index wheel coupled to the base pillar receiver.

In some embodiments, the percutaneous metatarsal fixation guide comprises a guide swivel interposed between the arm member and the drill guide, wherein the guide swivel permits rotation of the drill guide with respect to the guide end. In some embodiments, the guide swivel is a locking guide swivel. In some embodiments, the metatarsal fixation guide further comprises a second index wheel coupled to the drill guide.

Disclosed is a percutaneous metatarsal fixation guide comprising a pillar arm member having a pillar end; a guide arm member coupled to the pillar arm member and having a guide end; a base pillar receiver coupled to the pillar arm member proximate to the pillar end; and a drill guide coupled to the guide arm member proximate to the guide end.

In some embodiments, the pillar arm member and the guide arm member are coupled at a swivel joint. In some embodiments, the swivel joint is a locking swivel joint. In some embodiments, the pillar arm member and the base pillar receiver are coupled at a coronal swivel. In some embodiments, the pillar arm member and the guide arm member are coupled at a sagittal swivel.

Disclosed is a percutaneous metatarsal fixation guide comprising a pillar arm member having a pillar end; a coronal swivel coupled to the pillar arm member proximate to the pillar end, a base pillar receiver coupled to the coronal swivel; a guide arm member coupled to the pillar arm member at a swivel joint and having a guide end; a sagittal swivel coupled proximate to the guide end; and a drill guide coupled to the sagittal swivel, wherein the drill guide is adjustably positioned relative to the base pillar receiver by rotating the pillar arm at the coronal swivel in a coronal plane and rotating the drill guide at the sagittal swivel in a sagittal plane.

In some embodiments, the guide arm member is rotatably positioned with respect to the pillar arm member by causing rotation of the guide arm member at the swivel joint.

In some embodiments, the pillar arm member and the base pillar receiver are coupled at a coronal swivel. In some embodiments, the pillar arm member is rotatably positioned with respect to the base pillar receiver by causing rotation of the coronal swivel.

In some embodiments, the guide arm member and the drill guide are coupled at a sagittal swivel proximate to the guide end. In some embodiments, the drill guide is adjustably positioned relative to the base pillar receiver by causing rotation of the pillar arm at the coronal swivel in a coronal plane and causing rotation of the drill guide at the sagittal swivel in a sagittal plane.

Disclosed is a surgical instrument set comprising a percutaneous metatarsal fixation guide; a threaded fixation wire; and a non-threaded fixation wire.

Disclosed is a method for reduction and fixation of metatarsal fractures comprising steps mounting a percutaneous metatarsal fixation guide onto a base pillar inserted into a tarsal bone of a foot, aligning a drill sleeve of the metatarsal fixation guide with a central longitudinal axis of a fractured metatarsal bone, inserting a threaded fixation wire through a first insertion point located on a dorsal skin surface of a foot into two cortical surfaces of a distal fragment of a fractured metatarsal; distracting the distal fragment to a position beneath the insertion point by manipulating an external segment of the threaded fixation wire; reducing the distal fragment to a position contacting a proximal fragment of the fractured metatarsal; and fixing the reduced distal fragment to the proximal fragment,

In some embodiments, the fixing step comprises steps traversing a proximal end of a non-threaded fixation wire through a second insertion point located on a plantar skin surface of the foot, through the distal fragment, and across the fracture site near a central longitudinal axis of a metatarsal shaft of the proximal fragment; and coupling the non-threaded fixation wire to two cortical surfaces of a tarsometatarsal joint comprising a tarsal bone proximal to the fracture site, wherein the proximal end of the non-threaded fixation wire is positioned inside the tarsal bone. In some embodiments, the fixing step comprises traversing a proximal end of a non-threaded fixation wire through a second insertion point located on a plantar skin surface of the foot, through the distal fragment, and across the fracture site near a central longitudinal axis of a metatarsal shaft of the proximal fragment; passing the proximal end sequentially across a first cortical surface of a tarsometatarsal joint, a second cortical surface of the tarsometatarsal joint, and a dorsal skin surface of the foot, wherein the proximal end is positioned external to the dorsal skin surface of the foot; pulling the proximal end causing a distal end of the non-threaded fixation wire to retract through the second insertion point and into the distal fragment; and re-directing the distal end into subchondral cortical bone of the distal fragment.

In some embodiments, the method further comprises a step placing a cannulated screw over the non-threaded fixation wire, wherein the cannulated screw is coupled to the proximal fragment and the distal fragment.

The foregoing and other features and advantages of the present invention will be apparent from the following more detailed description of the particular embodiments of the invention, as illustrated in the accompanying drawings.

### BRIEF DESCRIPTION OF THE DRAWINGS

FIG. 1 is a perspective view of a percutaneous metatarsal fixation guide;

FIG. 2 is a perspective view of an alternative embodiment of a percutaneous metatarsal fixation guide;

FIG. 3a-b are perspective views of an additional alternative embodiment of a percutaneous metatarsal fixation guide;

FIG. 4a-c are perspective views of another alternative embodiment of a percutaneous metatarsal fixation guide;

FIG. 5 is a perspective view of an alternative embodiment of a percutaneous metatarsal fixation guide coupled to a tarsal bone;

FIG. 6 is a flowchart diagram of a method of reduction and fixation of a metatarsal fracture using a percutaneous metatarsal fixation guide; and

FIG. 7 is a flowchart diagram of an additional method of fixation of a metatarsal fracture using a percutaneous metatarsal fixation guide.

### DETAILED DESCRIPTION OF EMBODIMENTS OF THE INVENTION

The foregoing application describes a device and methods of use for a percutaneous metatarsal fixation guide. The metatarsal fixation guide provides an adjustable positioning guide for insertion of fixation hardware, such as a Kirshner wire (“K-wire”), across a distal metatarsal fracture, increasing the ease with which surgeon may achieve precise alignment of the distal and proximal bone fragments utilizing a percutaneous technique. Although application of the fixation guide to the treatment of metatarsal fractures is discussed in detail herein, this is not meant to be limiting. It is to be understood that the fixation guide may be used in percutaneous reduction and fixation of metacarpal fractures, and the like. With the fixation guide rigidly fixed to a tarsal bone through the dorsum of the foot in the desired position, the drill guide may be adjusted to align a fixation K-wire with the central longitudinal axis of the shaft of the fractured metatarsal. Adjustment of the fixation guide allows for adjustable positioning of the drill guide in the coronal, transverse, and sagittal planes relative to the drill guide.

The fixation guide is used for the performance of new surgical techniques for percutaneous reduction and fixation of metatarsal fractures, the detailed disclosures of which are provided herein. The new surgical techniques comprise use of a threaded K-wire inserted into the metatarsal head of a distal fracture fragment for use as a “toggle pin.” A surgeon uses the toggle pin to percutaneously manipulate the distal fragment into reduction with the proximal fragment at the fracture site. After the fracture is reduced, the fixation guide is rigidly coupled to the foot by clamping a “base pillar receiver” at or near one end of the fixation guide to a second threaded K-wire “base pillar” fixed into cortices of the tarsal bone articulating with the fractured metatarsal. A drill guide mounted opposite the fixation device from the base pillar receiver is used to insert a second K-wire across the reduced fracture. In some embodiments, index wheels at either end of the fixation device bearing corresponding markings are used to match a radiopaque alignment guide coupled to the index wheel at the base pillar and of the guide with the long axis of a drill sleeve coupled to the index wheel at the guide end of the fixation guide. Following alignment of the radiopaque alignment guide with the central longitudinal axis of the fractured metatarsal under fluoroscopy, the long axis of the drill sleeve can be matched in three planes to the central longitudinal axis of the fractured metatarsal. Placement of fixation hardware correctly aligned with the central longitudinal axis of the fractured metatarsal is, therefore, greatly facilitated.

Various embodiments of the device may include a uniplanar or bi-planar swivel joint located at the base pillar fixation end and the drill guide end, wherein the swivel planes are oriented normal to one another. With this mechanism, an operator, such as a foot surgeon, may precisely adjust the position and angle of the drill guide with respect to a central longitudinal axis of the fractured metatarsal's shaft such that the surgeon may position fixation hardware to maintain the reduced proximal and distal fragments in alignment for healing.

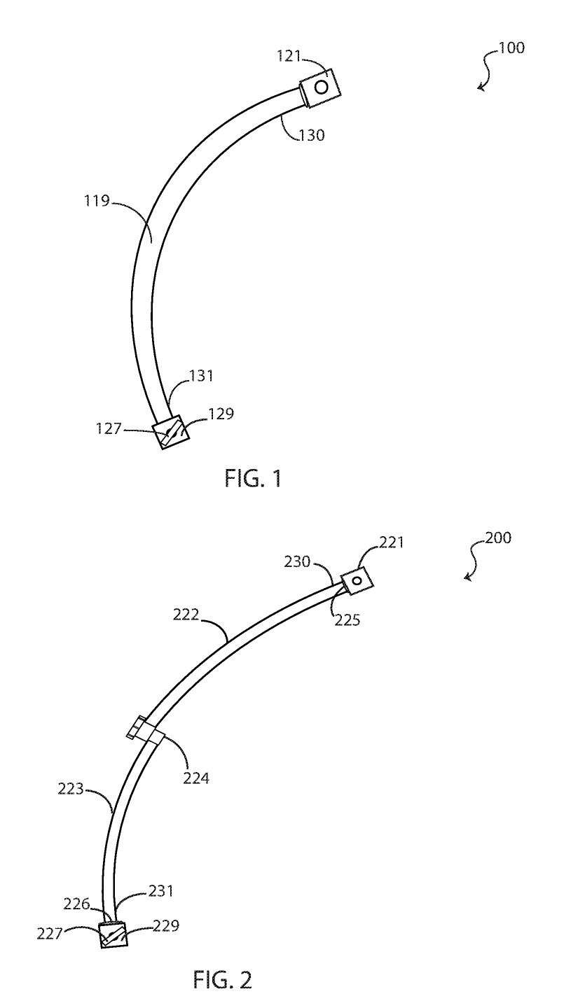

FIG. 1 is a perspective view of a percutaneous metatarsal fixation guide. FIG. 1 shows a percutaneous metatarsal fixation guide 100 comprising an arm member 119. Arm member 119 is a curvilinear member having two ends opposite one another a pillar end 130 and a guide end 131. In some embodiments (not shown in the drawing figures), arm member 119 comprises a series of rectilinear segments formed as a unitary body. In some embodiments, including the embodiment shown in FIG. 2 and described in detail herein below, arm member 119 comprises two curvilinear members coupled at a central swivel. Arm member 119 is shaped in a manner wherein an operator may simultaneously position pillar end 130 over the dorsum of the hindfoot of a patient with a metatarsal fracture and guide end 131 over the plantar surface of the patient's forefoot. This positioning allows a user of percutaneous metatarsal fixation guide 100, such as a foot surgeon or other healthcare provider, to externally align fixation guide 100 with the proximal (base) of the fractured metatarsal and the distal (head) of the fractured metatarsal bone prior during reduction of the fracture and insertion of fixation hardware.

FIG. 1 additionally shows a base pillar receiver 121 coupled to pillar end 120 and a drill guide 129 coupled to guide end 131. Although base pillar receiver 121 and drill guide 129 are shown coupled at pillar end 120 and guide end 131 of arm member 119 respectively, this is not meant to be limiting. In some embodiments (not shown), base pillar receiver 121 is coupled proximate to pillar end 120 but not directly at pillar end 120. Similarly, drill guide 129, in some embodiments, is coupled proximate to guide end 131 but not direction at guide end 131.

In some embodiments, base pillar receiver 121 is fixedly coupled to pillar end 120 and in other embodiments, guide pillar receiver 121 is adjustably coupled to arm member 119 proximate to pillar end 130, wherein a user of fixation guide 100 may adjust the position of guide pillar receiver 121 along arm member 119 proximate to pillar end 120 by sliding guide pillar receiver 121 along a length of arm member 119. The ability to slide guide pillar receiver 121 for at least a limited distance along arm member 119 may facilitate the use of metatarsal fixation guide 100 in treating metatarsal fractures in individuals with different sized feet, for example. Following positioning of guide pillar receiver 121 on arm member 119, a locking means (not shown) for holding guide pillar receiver 121 in position, may be activated by the user to fix guide pillar receiver 121 in position on arm member 119 relative to pillar end 130. The locking means may comprise a friction/set screw, a spring-actuated stop on base pillar receiver 121 that reversibly engages with a surface feature of arm member 119, or the like.

In a similar fashion, in some embodiments, drill guide 129 is adjustably coupled to arm member 119 proximate to guide end 131, wherein a user may adjust the position of drill guide 129 along arm member 119 relative to guide end 131 and base pillar receiver 121 to accommodate the use of metatarsal fixation guide 100 in treating metatarsal fractures on different individuals having a range of different foot sizes. In some embodiments, an adjustably positioned drill guide 129 is reversibly fixed on arm member 119 by a locking means, such as a set screw or the like. Additionally, drill guide 129 is rotatably coupled to arm member 119, wherein a user of fixation guide 100 may rotate drill guide 120 in a parasagittal plane to align the long axis (not shown) of drill guide 129 with a central longitudinal axis of the fractured metatarsal bone.

FIG. 2 is a perspective view of an alternative embodiment of a percutaneous metatarsal fixation guide. FIG. 2 shows a metatarsal fixation guide 200 comprising a pillar arm member 222 and a guide arm member 223 coupled opposite pillar end 230 and guide end 231 by a central swivel 224. Central swivel 224 allow fixation guide 200 to be collapsed into a smaller profile for storage when not in use, and to be opened-up when ready for use. In some embodiments, central swivel 224 locks in either a closed position or an open position. The closed position means a position wherein pillar arm member 222 and guide arm member 223 are folded together against one another. The open position means a position wherein pillar arm member 222 and guide arm member 223 are about coplanar, forming an arc in the general shape of metatarsal fixation guide 200 shown in FIG. 2.

Additionally and similar to arm member 119 of fixation guide 100, pillar arm member 222 a base pillar receiver 222 coupled proximate to pillar end 230 and guide arm member 223 comprises a drill guide 229 coupled proximate to guide end 231.

In the embodiment shown by FIG. 2, and in some other embodiments, fixation guide 200 comprises a coronal swivel 226 coupled to pillar arm member 222 proximate to pillar end 130. Coronal swivel 226 is additionally coupled to base pillar receiver 221, wherein base pillar receiver 221 may rotate in a coronal plane by operation of coronal swivel 225. Rotation of base pillar receiver 221 in the coronal plane, in some embodiments, facilitates movement of pillar arm member 222 and guide arm member 223 in the coronal plane with respect to base pillar receiver 221, which is fixed in space when coupled to a base pillar, as discussed in detail herein below. Additionally, a guide swivel 226 is coupled to guide arm member 223, in some embodiments, proximate to guide end 231 and additionally coupled to drill guide 229, in some embodiments. Guide swivel 226 allows an operator to rotate drill guide 229 in a transverse plane relative to the proximal fragment of the fractured metatarsal, 90° to the coronal plane. In some embodiments wherein metatarsal fixation guide 200 comprises both coronal swivel 225 and guide swivel 226, therefore, alignment of drill guide 129 with the proximal and distal metatarsal fracture fragments can be adjusted in the coronal plane independently in all three planes—sagittal, coronal, and transverse. This relationship is important because drill guide 229 may be adjustably positioned relative to base pillar receiver 221 by rotating pillar arm member 222 at the coronal swivel in a coronal plane while independently rotating drill guide 229 at guide swivel 226 in the transverse plane. The independent rotation of guide end 231 in the sagittal plane with respect to the coronal plane permits an operator to align drill guide 229 in three planes (coronal, sagittal, and transverse), allowing precise placement of a K-wire across the fracture site and through the tarsometatarsal joint generally along the central longitudinal metatarsal axis.

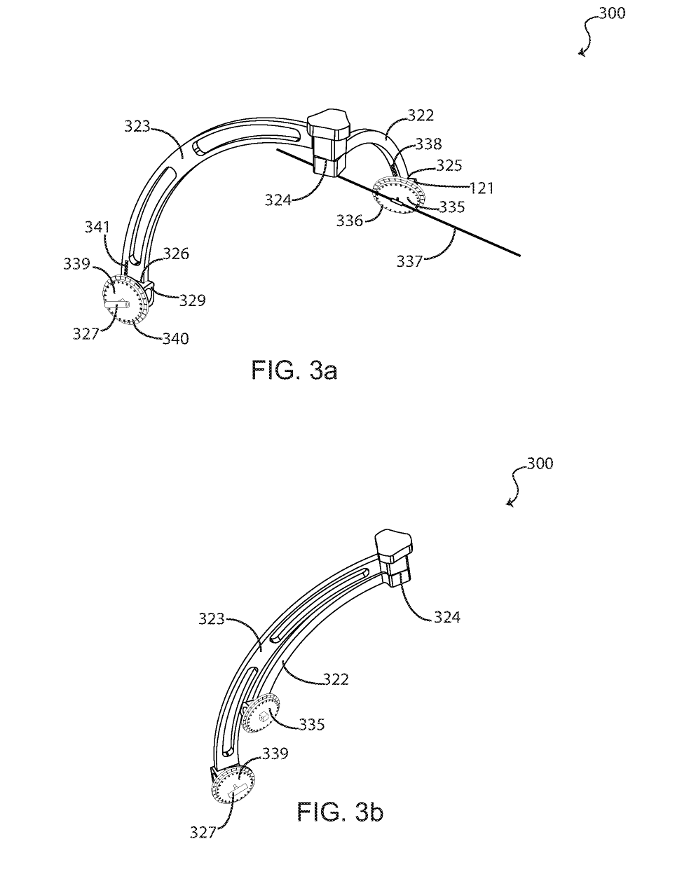

FIG. 3a and FIG. 3b are perspective views of an additional alternative embodiment of a percutaneous metatarsal fixation guide. FIGS. 3a-b show a metatarsal fixation guide 300 comprising a pillar arm member 322 and a guide arm member 323 coupled by a central swivel 324. In some embodiments, including the embodiment shown in FIGS. 3a-b, a coronal swivel 325 couples a base pillar receiver 321 to pillar arm member 121 wherein a user may rotate base pillar receiver 321 in the coronal plane with respect to pillar arm member 322. Additionally, a guide swivel 326 couples a drill guide 329 to the guide arm member 323, wherein a user may rotate a drill guide 329 in the transverse plane with respect to guide arm member 323, in some embodiments.

In some embodiments, metatarsal fixation guide 300 additionally comprises a first index wheel 335 and a second index wheel 339, wherein the first and second index wheels provide a means wherein the user of fixation guide 300 may precisely align fixation hardware inserted via drill sleeve 227 with the central longitudinal axis of the fractured metatarsal. As shown in FIG. 3a, first index wheel 335 is rotatably coupled to base pillar receiver 321 and second index wheel 339 is rotatably coupled to drill guide 329. First index wheel 335 bears a plurality of first index markings 336 and second index wheel 329 bears a plurality of second index markings 340. A first fixed reference point 338 is displayed on pillar arm member 322 and a second fixed reference point 341 is displayed on guide arm 323.

In some embodiments wherein first index wheel 335 is coupled to base pillar receiver 321, first index wheel 335 is also non-rotationally coupled to an alignment guide 337. Alignment guide 337 is a generally elongate member, such as a thin rod, K-wire, or the like and is formed from a radiopaque material wherein alignment guide 337 is visible radiographically, such as on static x-ray or intraoperative fluoroscopy. In embodiments wherein index wheel 335 is rotatably coupled to drill guide 329, index wheel 335 is also non-rotationally coupled to drill sleeve 327. In some embodiments, alignment guide 337 is fixedly coupled to first index wheel 335. In some embodiments alignment guide 337 is removably coupled to first index wheel 335.

Notably, alignment guide 137 is coupled to first index wheel 335 with the same orientation to first index markings 136 as drill sleeve 327 of second index wheel 339 has to second index markings 340. In other words, under a condition wherein first index wheel 335 is rotated to a position wherein a first index marking 336 “Q,” for example, lines up with first fixed reference point 338 and a corresponding second index marking 340 “Q” is rotated to a corresponding position lined up with second fixed reference point 341, alignment guide 337 is generally parallel to drill sleeve 327. First index wheel 335 and second index wheel 339 are utilized by the user of some embodiments of metatarsal fixation guide 300 to precisely align fixation hardware (not shown in FIGS. 3a-b) inserted through drill sleeve 327 with the central longitudinal axis of the fractured metatarsal bone.

A foot surgeon using fixation guide 300 percutaneously fixed to a hindfoot bone (see detailed discussion herein below) may rotate first index wheel 335 under intraoperative fluoroscopy until the observed position of alignment guide 337 is generally parallel to the central longitudinal axis of the fractured metatarsal and then note which first index marking 336 lines up with first fixed reference point 338. The surgeon then simply rotates second index wheel 335 until the corresponding second index marking 340 lines up with second fixed reference point 341. Drill sleeve 327 is then generally parallel to the central longitudinal axis of the fractured metatarsal, enabling the surgeon to percutaneously place fixation hardware, such as a K-wire, through the drill sleeve into the patient's foot and through the central longitudinal axis, facilitating accurate alignment and fixation of the bone fragments comprising the reduced metatarsal fracture.

First index wheel 335 and second index wheel 339 are each centrally mounted on a pin, sleeve bearing, axle, or similar structure coupled to base pillar receiver 321 or drill guide 329 respectively. Each index wheel may be rotated on its corresponding pin or axle independent of the other index wheel. Rotation of first index wheel 335 causes a corresponding rotation of alignment guide 337 and rotation of second index wheel 339 causes a corresponding rotation of drill sleeve 327, however, as can be appreciated from FIG. 3a. Moreover, the plurality of first index markings 336 are disposed on first index wheel 335 in the same order from a first fixed reference point 338 borne on pillar arm member 322 as the plurality of second index markings 340 borne on guide arm member 323. Because first index markings 336 on first index wheel 335 correspond with matching index markings 336 on the second index wheel 335, alignment of a particular index marking 336 on index wheel 335 with the fixed reference point 338 and alignment of the particular corresponding index marking 336 of second index wheel 335 with second fixed reference point 338 causes drill sleeve 327 to be oriented parallel to alignment guide 337.

First index markings 336 and second index markings 340 may comprise letters of the alphabet, such as “A”-“Z,”, or numbers, such as “1”-“26,” for example. Individual index markings are equally spaced around a perimeter of first index wheel 335 and second index wheel 339. For example, in embodiments of fixation guide 300 comprising 24 individual first index markings 336 and 24 individual second index markings 340, the individual index markings are disposed each fifteen (15) degrees about the perimeter of each of first index wheel 335 and second index wheel 339.

FIG. 3b is a perspective view of fixation guide 300 in a collapsed position. A central swivel 324 allows pillar arm member 322 and guide arm member 323 to nest together, facilitating storage of fixation guide 300. In some embodiments, central swivel 324 locks in a first “open” position wherein pillar arm member 322 and guide arm member 323 are reversibly fixed in a shape that generally forms an arc, similar to the shape of pillar arm member 222 and guide arm member 223 shown in FIG. 2, and also locks in a second “closed” position fixed in the shape shown in FIG. 3b. In some embodiments, central swivel locks into any position along a continuum of positions between the first open position and the second closed position, such as the position shown in FIG. 3a, for example.

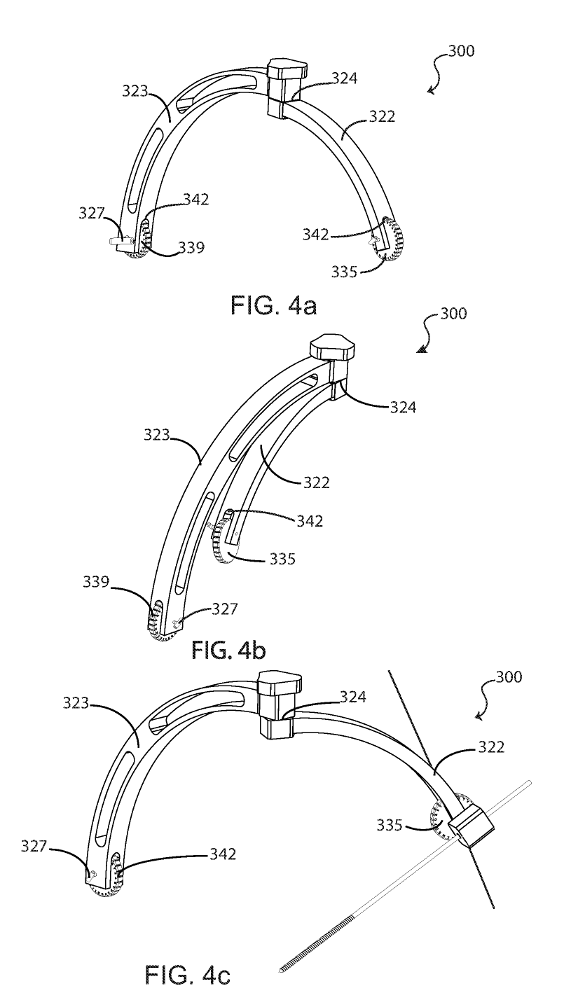

FIGS. 4a-c are a perspective view of an alternative embodiment of percutaneous metatarsal fixation device 300. FIG. 4a shows fixation device 300 in first fully open position, FIG. 4b shows fixation device 300 second closed position, and FIG. 4c shows fixation device 300 an intermediate position. Additionally, the embodiment of fixation device 300 shown in FIGS. 4a-c comprises first index wheel 335 and second index wheel 339 each mounted on an axle within a receiving slot 342, wherein first index wheel 335 is generally coplanar with pillar arm 322 and second index wheel 339 is generally coplanar with guide arm 323.

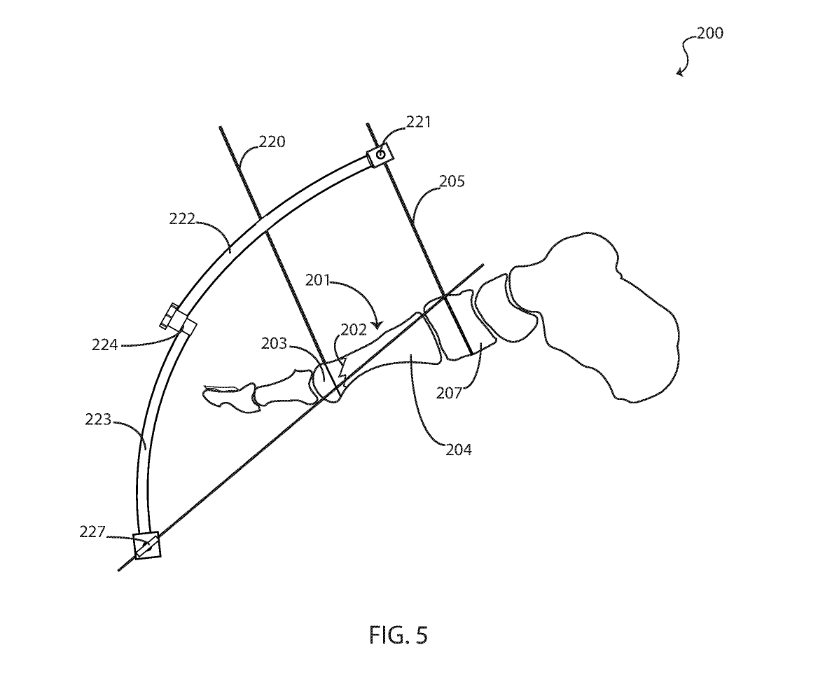

FIG. 5 is a perspective view of an alternative embodiment of a percutaneous metatarsal fixation guide coupled to a tarsal bone. FIG. 5 shows a distal fragment 203 and a proximal fragment 204 reduced together at a fracture site 202 of a metatarsal bone 101. The surgeon reduces the fracture by inserting a threaded K-wire for use as a “toggle pin” through the dorsum of the foot into two cortices of the metatarsal head portion of distal fragment 203. FIG. 5 shows toggle pin 220 coupled to distal fragment 203, following insertion. Following insertion into distal fragment 203, the foot surgeon using fixation device 200 may grasp toggle pin 220 and manipulate distal fragment 203, generally under fluoroscopic or other radiographic guidance, to reduce distal fragment 203 and proximal fragment 204 at fracture site 202. A detailed description of steps associated with reducing fracture site 202 using toggle pin 220 is presented in detail under discussion of method 400 herein below.

FIG. 5 additionally shows a base pillar 205 inserted into a tarsal bone 207. Base pillar 205 is a stanchion rigidly fixed to tarsal bone 207 that provides a point of support and fixation for metatarsal fixation guide 200. Tarsal bone 107 is a tarsal bone from the group of tarsal bones comprising a medial cuneiform, a middle cuneiform, a lateral cuneiform, or a cuboid bone. Tarsal bone 107 articulates with the base of the fractured metatarsal, therefore, the particular tarsal bone comprising tarsal bone 107 depends on which metatarsal bone is fractured. Base pillar 205 may be a threaded rigid wire or rod, commonly a threaded K-wire, which is inserted percutaneously through the dorsum of the foot generally perpendicular to the dorsal cortical surface of the tarsal bone and passed through two cortical surfaces, resulting in a rigid coupling of base pillar 205 to tarsal bone 207. Fixation device 200 is then reversibly coupled to base pillar 205 at base pillar receiver 221 by a reversible coupling means, such as a set screw or the like.

The components defining any metatarsal fixation device may be formed of any of many different types of materials or combinations thereof that can readily be formed into shaped Objects provided that the components selected are consistent with the intended operation of a metatarsal fixation device. For example, the components may be formed of: rubbers (synthetic and/or natural) and/or other like materials; glasses (such as fiberglass) carbon-fiber, aramid-fiber, any combination thereof, and/or other like materials; polymers such as thermoplastics (such as ABS, Fluoropolymers, Polyacetal, Polyamide; Polycarbonate, Polyethylene, Polysulfone, and/or the like), thermosets (such as Epoxy, Phenolic Resin, Polyimide, Polyurethane, Silicone, and/or the like), any combination thereof, and/or other like materials; composites and/or other like materials; metals, such as zinc, magnesium, titanium, copper, iron, steel, carbon steel, alloy steel, tool steel, stainless steel, aluminum, any combination thereof, and/or other like materials; alloys, such as aluminum alloy, titanium alloy, magnesium alloy, copper alloy, any combination thereof, and/or other like materials; any other suitable material; and/or any combination thereof.

Furthermore, the components defining any metatarsal fixation guide may be purchased pre-manufactured or manufactured separately and then assembled together. However, any or all of the components may be manufactured simultaneously and integrally joined with one another. Manufacture of these components separately or simultaneously may involve extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled with one another in any manner, such as with adhesive, a weld, annealing, a fastener (e.g. a bolt, a nut, a screw, a nail, a rivet, a pin, and/or the like), wiring, any combination thereof, and/or the like for example, depending on, among other considerations, the particular material forming the components. Other possible steps might include sand blasting, polishing, powder coating, zinc plating, anodizing, hard anodizing, and/or painting the components for example.

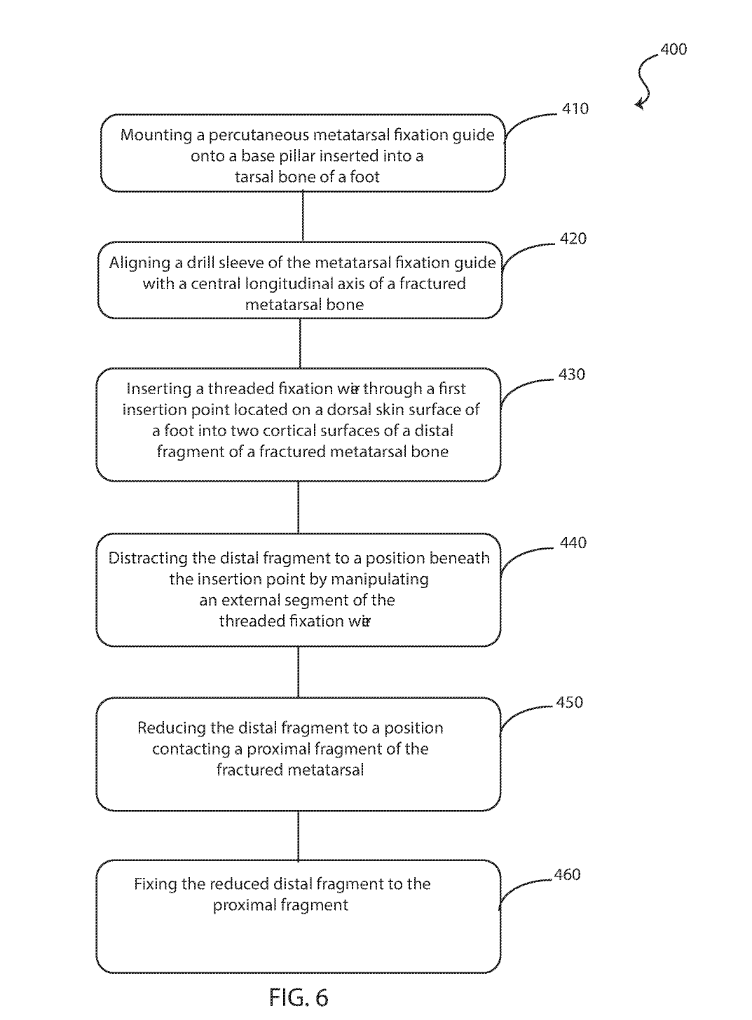

FIG. 6 is a flowchart diagram of a method of reduction and fixation of a metatarsal fracture using a percutaneous metatarsal fixation guide. FIG. 6 shows a method 400 comprising a mounting step 410, an aligning step 420, an inserting step 430, a distracting step, a reducing step 450, and a fixing step 460.

A percutaneous metatarsal fixation guide is use by a surgeon, such as an orthopedist or a podiatrist, for example. In preparation for performing method 400, the user of the fixation guide obtains standard multi-view radiographs of the foot comprising the fractured metatarsal bone. Under anesthesia, the surgeon examines the fracture site dorsally and ventrally by palpation.

Mounting step 410, in some embodiments, comprises mounting a percutaneous metatarsal fixation guide onto a base pillar inserted into a tarsal bone of a foot. A base pillar is fixed to a hindfoot bone and serves as the anchor for the fixation guide and the reference point for alignment of fixation hardware placement. In some embodiments, the base pillar is a threaded fixation wire, such as a 1.6-millimeter threaded Kirshner-wire, or the like. A stab incision is made in the skin of the dorsum of the foot centered over the distal aspect of the tarsal bone articulating with the fractured metatarsal and blunt dissection is used to expose the periosteum, just proximal to the tarsometatarsal joint. Depending upon which metatarsal bone is fractured, the hindfoot/tarsal bone may be the cuboid or one of the three cuneiform bones. The foot surgeon then loads the base pillar into a drill or other standard driving device and inserts the base pillar through the stab incision, driving the pillar through the tarsal bone until the upper-most thread (“run-out”) of the threaded wire is flush with the subcortical surface of the dorsal cortex of the tarsal bone. The run-out thereafter serves as a precise radiographic reference point for subsequent placement of a fixation wire, or other fixation hardware. The percutaneous metatarsal fixation device may comprise two arms a pillar arm and a guide arm or, alternatively, a single arm with a pillar end and a guide end. Either the pillar arm or the pillar end of the single arm is reversibly mounted on the base pillar, such as with a set screw or similar reversible mounting means. In some embodiments, a central swivel of the fixation guide coupling the pillar arm to the guide arm may be adjusted at an oblique angle, if desired. A removable alignment guide is then inserted into an index wheel of the pillar-mounted fixation guide, in some embodiments.

Aligning step 420, in some embodiments, comprises aligning a drill sleeve of the metatarsal fixation guide with a central longitudinal axis of a fractured metatarsal bone. The fixation guide, in some embodiments, has an interchangeable drill sleeve mounted to the guide arm or the guide end of the single arm to accommodate fixation hardware of different diameters and sizes. Intraoperative x-ray, such as intraoperative fluoroscopy, is used to visualize a central longitudinal axis of the fractured metatarsal bone, wherein the surgeon rotates a first index wheel bearing the alignment guide until the alignment guide is generally parallel to the central longitudinal axis. The surgeon then notes a first index marking corresponding to a first fixed reference point at the pillar end or pillar arm and rotates a second index wheel coupled to the guide end or the guide arm until a corresponding second index marking on the second index wheel aligns with a second fixed reference point at the guide end or the guide arm. This maneuver brings a drill sleeve coupled to the second index wheel generally parallel to the alignment guide. The drill sleeve is, therefore, generally parallel to the central longitudinal axis of the fractured metatarsal bone.

Inserting step 430, in some embodiments, comprises inserting a threaded fixation wire through a first insertion point located on a dorsal skin surface of a foot into two cortical surfaces of a distal fragment of a fractured metatarsal bone. The inserted fixation wire is later used as a handle or “toggle pin” for the surgeon to manipulate the distal fragment, reducing the distal fragment onto the proximal fragment. While palpating the head of the fractured metatarsal through the plantar surface of the foot, the surgeon positions a partially threaded K-wire through a stab incision through the skin dorsal to the location of the fracture site. A 1.6-millimeter K-wire is an appropriate size, however a slightly larger or slightly small gauge wire may be used, depending upon the gender, age, and physical size of the patient and the particular metatarsal (i.e., first versus fifth) that is fractured. The surgeon aims the fixation wire at the plantarly palpated metatarsal head (distal fracture fragment) in the coronal plane of a three-dimensional fracture site oblique to the sagittal and transverse planes. The surgeon then drives the fixation wire through both cortices of the metatarsal head of the distal fracture fragment using a wire driver.

Distracting step 440, in some embodiments, comprises distracting the distal fragment to a position beneath the insertion point by manipulating an external segment of the threaded fixation wire. The surgeon uses the fixation wire as a “toggle pin,” pulling on the wire to distract the distal fragment into a subcutaneous position along the path the fragment traveled from the origin of the fracture site. This maneuver will bring the distal fragment just below the percutaneous insertion point of the “toggle pin,” allowing movement in all three cardinal planes (sagittal, coronal, and transverse) within the joint capsule. The maneuver will also allow rotation in the transverse plane around a vertical axis as well as some sagittal plane rotation around a horizontal axis, depending on the obliquity of the “toggle pin.” The more horizontal the axis, the more sagittal plane rotation and plantar flexion can be reduced.

Reducing step 450, in some embodiments, comprises reducing the distal fragment to a position contacting a proximal fragment of the fractured metatarsal. Reducing step 450 may be performed under intra-operative fluoroscopy. The surgeon stands in front of the foot (at the end of the operating table), grasps the toggle pin, and moves the distal fragment into reduction against the proximal fragment at the fracture site. Reduction of the fracture, with proper alignment, may be confirmed with plain anteroposterior and lateral plain x-rays, prior to fixing step 440.

Fixing step 440, in some embodiments, comprises fixing the reduced distal fragment to the proximal fragment. Fixing step 440 may be performed by any conventional fixation means, currently known in the art, or which may be developed anytime in the future. Of note, fixing step 440 employs use of the drill sleeve aligned generally parallel with the central longitudinal axis for alignment of the fixation hardware versus “free-handing” the fixation wire or other hardware across a fracture site of a relatively small metatarsal bone, which may be technically difficult and imprecise.

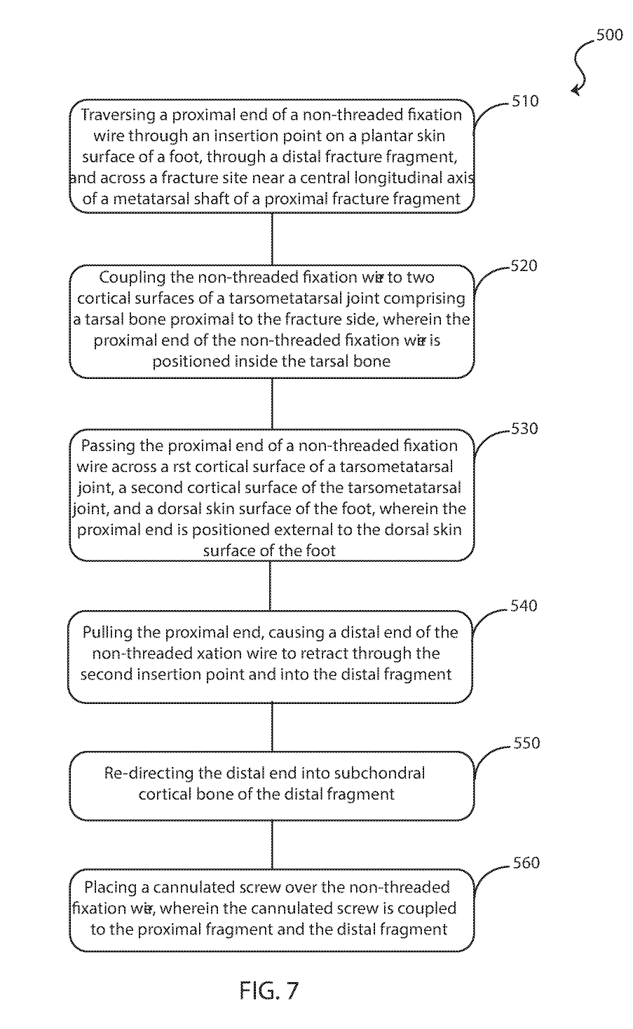

FIG. 7 is a flowchart diagram of a method of fixing a fractured metatarsal using a percutaneous metatarsal fixation guide. Fixing method 500 is performed following method 400, wherein fixation hardware is placed across the reduced and aligned fracture site caused by performing the steps of method 400. Fixing method 500, in some embodiments, comprises a traversing step 510, a coupling step 520, and a passing step 530. In some embodiments, method 500 additionally comprises a pulling step 540. In some embodiments, method 500 additionally comprises a re-directing step 550. In some embodiments, method 500 additionally comprises a placing step 560.

Traversing step 510, in some embodiments, comprises traversing a proximal end of a non-threaded fixation wire through an insertion point on a plantar skin surface of a foot, through a distal fracture fragment, and across a fracture site near a central longitudinal axis of a metatarsal shaft of a proximal fracture fragment. A foot surgeon may hold a toggle pin in one hand while an assistant dorsiflexes the digit, enabling the foot surgeon to position a non-threaded fixation wire, such as a 1.6 millimeter smooth Kirshner wire, for example, through the drill sleeve aligned with the central longitudinal axis, and into the plantar-aspect of the metatarsal head, as close to the central longitudinal axis of the fractured metatarsal as possible. Lateral viewing of the fracture site using intraoperative fluoroscopy facilitates positioning of the fixation wire. After the surgeon confirms proper positioning, the surgeon uses the anteroposterior fluoroscopic view and drives the fixation wire across the fracture site while aiming for the base of the proximal fragment of the fractured metatarsal. The surgeon traverses the fixation wire across the fracture site and continues along the central longitudinal axis into the base of the fractured metatarsal.

Coupling step 520, in some embodiments, comprises coupling the non-threaded fixation wire to two cortical surfaces of a tarsometatarsal joint comprising a tarsal bone proximal to the fracture site, wherein the proximal end of the non-threaded fixation wire is positioned inside the tarsal bone. After completion of traversing step 510, the surgeon continues driving the fixation K-wire across the cortex of the metatarsal base, across the tarsometatarsal joint, and into a tarsal bone; i.e., the cuboid or a cuneiform bone, depending on which particular tarsal bone comprise the proximal aspect of the tarsometatarsal joint of the fractured metatarsal. In is generally important to pass the fixation wire through two stable cortices proximal to the fracture site. It may also be useful, during coupling step 520, to grasp the toggle pin to stabilize the reduced fracture by applying counter-pressure to maintain proper reduction, bone length, and alignment. In some embodiments, reduction and alignment is verified using intraoperative plain x-rays.

In some embodiments, fixing method 500 additionally comprises a passing step 530. Passing step 530, in some embodiments, comprises passing the proximal end of the non-threaded fixation wire across a first cortical surface of the tarsometatarsal joint, a second cortical surface of the tarsometatarsal joint, and a dorsal skin surface of the foot, wherein the proximal end is positioned external to the dorsal skin surface of the foot. If reduction and alignment appear sub-optimal on plain films, further adjustments can be made. For example, there is usually minimal coronal plane rotation mal-alignment due to the soft tissue attachments about the plantar plant. However, if there is significant coronal plane rotational mal-alignment, the surgeon can temporarily remove the toggle pin and use the fixation wire to rotate the distal fragment about its longitudinal axis. After coupling is complete, the surgeon may bend, cut, and cap the exposed ends of the fixation wire on the plantar forefoot and dorsal hindfoot near the level of the skin.

In some embodiments, fixing method 500 additionally comprises a pulling step 540. Pulling step 540, in some embodiments, comprises pulling the proximal end, causing a distal end of the non-threaded fixation wire to retract through the second insertion point and into the distal fragment. For example, the dorsally exposed end of the fixation wire may be pulled further proximally until the plantar end is retracted through the plantar skin insertion site and, subsequently, into the subchondral bone of the metatarsal head, where it becomes firmly seated. Pulling step 54, in some embodiments, is performed under fluoroscopy to enable proper placement of the plantar end of the fixation wire in the subchondral bone of the metatarsal head of the distal fracture fragment.

Fixing method 500 may also comprise re-directing step 550. Redirecting step 550, in some embodiments, comprises re-directing the distal end of the fixation wire into subchondral cortical bone of the distal fragment. In some embodiments, redirecting step 550 may be performed by holding the toggle pin to stabilized the reduce distal fragment and provide counter pressure while pulling the fixation wire further retrograde into the fracture site, and the re-directing slightly plantar and driving the fixation wire back antegrade into the metatarsal head of the distal fragment, entering “un-breached” subchondral cortical bone in a position slightly plantar to the retrograde insertion site of the fixation wire in the distal fragment. Care is taken not to drive the plantar end of the fixation wire through cortical bone and into the metatarsophalangeal joint. Pulling step 540 may be completed under fluoroscopic guidance, with confirmation of proper reduction, alignment, post-reduction bone length, and fixation wire positioning using intraoperative plain x-rays.

Method 500 may further comprise a placing step 560. Placing step 560, in some embodiments, comprises placing a cannulated screw over the non-threaded fixation wire, wherein the cannulated screw is coupled to the proximal fragment and the distal fragment. A small stab incision may be made dorsally in line with the percutaneous fixation wire followed by blunt dissection down to the dorsal surface of the tarsal bone. The fixation wire is then measured for placement of a cannulated screw thrown from dorsal-proximal to plantar-distal, ending in the strong, subchondral bone of the metatarsal head, taking care not to penetrate the plantar aspect of the metatarsophalangeal joint. The fixation wire may then be removed by retracting prograde through the skin insertion point on the dorsum of the foot, and the cannulated screw head counter-sunk flush with the dorsal cortical surface of the tarsal bone. Alternatively, the cannulated screw may be thrown from plantar-distal to dorsal-proximal, ending in the subchondral bone of the metatarsal base. A percutaneous headless 4.0 cannulated screw may be used. It is important for the cannulated screw to be of sufficient length to purchase subchondral cortical bone at both the base and the metatarsal head of the fractured metatarsal, to create compression at the reduced fracture site.

Performance of method 400, method 500, or method 400 and method 500 are not limited to reduction and fixation of a fractured metatarsal bone. A percutaneous fixation guide, including some embodiments of the percutaneous metatarsal fixation guide describe herein, may be used according to the steps of methods 400 and 500 to percutaneously reduce and fix fractures of the metacarpal bones, for example. None of the examples of the design or use of a percutaneous metatarsal fixation guide recited herein is intended to be limiting with respect to metatarsal fractures.

A metatarsal fixation guide, including a method of use in reduction and fixation of metatarsal fractures, is disclosed. The metatarsal fixation guide and method of use overcome deficiencies of the existing art by allowing a foot surgeon to reduce and then accurately fix a proximal and distal fragment of a fractured metatarsal in proper alignment using fixation hardware. The fixation guide bearing two corresponding index wheels is rigidly mounted to a tarsal bone via a temporary threaded K-wire, or similar hardware, wherein an index marking on a second index wheel coupled to a drill sleeve may be positioned to correspond with an index marking on first index wheel coupled to an alignment guide, such that the drill sleeve is aligned with the central longitudinal axis of the fractured metatarsal. This allows precise placement of fixation hardware, compared to using a conventional “free-hand” technique.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

What is claimed is:

1. A percutaneous metatarsal fixation guide comprising:
an arm member having a pillar end and a guide end;
a base pillar receiver coupled to the arm member proximate to the pillar end; and
a drill guide coupled to the arm member proximate to the guide end.

2. The percutaneous metatarsal fixation guide of claim 1, further comprising a coronal swivel interposed between the arm member and the base pillar receiver, wherein the coronal swivel permits rotation of the base pillar receiver with respect to the pillar end.

3. The percutaneous metatarsal fixation guide of claim 2, wherein the coronal swivel is a locking coronal swivel.

4. The percutaneous metatarsal fixation guide of claim 2, further comprising a first index wheel coupled to the base pillar receiver.

5. The percutaneous metatarsal fixation guide of claim 1, further comprising a guide swivel interposed between the arm member and the drill guide, wherein the guide swivel permits rotation of the drill guide with respect to the guide end.

6. The percutaneous metatarsal fixation guide of claim 5, wherein the guide swivel is a locking guide swivel.

7. The percutaneous metatarsal fixation guide of claim 5, further comprising a second index wheel coupled to the drill guide.

8. A percutaneous metatarsal fixation guide comprising:
a pillar arm member having a pillar end;
a guide arm member coupled to the pillar arm member and having a guide end;
a base pillar receiver coupled to the pillar arm member proximate to the pillar end; and
a drill guide coupled to the guide arm member proximate to the guide end.

9. The percutaneous metatarsal fixation guide of claim 8, wherein the pillar arm member and the guide arm member are coupled at a swivel joint.

10. The percutaneous metatarsal fixation guide of claim 9, wherein the swivel joint is a locking swivel joint.

11. The percutaneous metatarsal fixation guide of claim 9, wherein the guide arm member is rotatably positioned with respect to the pillar arm member by causing rotation of the guide arm member at the swivel joint.

12. The percutaneous metatarsal fixation guide of claim 8, wherein the pillar arm member and the base pillar receiver are coupled at a coronal swivel.

13. The percutaneous metatarsal fixation guide of claim 12, wherein the pillar arm member is rotatably positioned with respect to the base pillar receiver by causing rotation of the coronal swivel.

14. The percutaneous metatarsal fixation guide of claim 8, wherein the guide arm member and the drill guide are coupled at a sagittal swivel proximate to the guide end.

15. The percutaneous metatarsal fixation guide of claim 14, wherein the drill guide is adjustably positioned relative to the base pillar receiver by causing rotation of the pillar arm at the coronal swivel in a coronal plane and causing rotation of the drill guide at the sagittal swivel in a sagittal plane.

16. A method for reduction and fixation of metatarsal fractures comprising steps:
mounting a percutaneous metatarsal fixation guide onto a base pillar inserted into a tarsal bone of a foot;
aligning a drill sleeve of the metatarsal fixation guide with a central longitudinal axis of a fractured metatarsal bone;
inserting a threaded fixation wire through a first insertion point located on a dorsal skin surface of a foot into two cortical surfaces of a distal fragment of a fractured metatarsal;
distracting the distal fragment to a position beneath the insertion point by manipulating an external segment of the threaded fixation wire;
reducing the distal fragment to a position contacting a proximal fragment of the fractured metatarsal; and
fixing the reduced distal fragment to the proximal fragment.

17. The method of claim 10, wherein the fixing step comprises steps:
traversing a proximal end of a non-threaded fixation wire through a second insertion point located on a plantar skin surface of the foot, through the distal fragment, and across the fracture site near a central longitudinal axis of a metatarsal shaft of the proximal fragment; and
coupling the non-threaded fixation wire to two cortical surfaces of a tarsometatarsal joint comprising a tarsal bone proximal to the fracture site, wherein the proximal end of the non-threaded fixation wire is positioned inside the tarsal bone.

18. The method of claim 10, wherein the fixing step comprises:
traversing a proximal end of a non-threaded fixation wire through a second insertion point located on a plantar skin surface of the foot, through the distal fragment, and across the fracture site near a central longitudinal axis of a metatarsal shaft of the proximal fragment;
passing the proximal end sequentially across a first cortical surface of a tarsometatarsal joint, a second cortical surface of the tarsometatarsal joint, and a dorsal skin surface of the foot, wherein the proximal end is positioned external to the dorsal skin surface of the foot;
pulling the proximal end causing a distal end of the non-threaded fixation wire to retract through the second insertion point and into the distal fragment; and
re-directing the distal end into subchondral cortical bone of the distal fragment.

19. The method of claim 10, further comprising a step placing a cannulated screw over the non-threaded fixation wire, wherein the cannulated screw is coupled to the proximal fragment and the distal fragment.
